(19) United States
(12) Patent Application Publication
(10) Pub. No.: US 2026/0060586 A1
(43) Pub. Date: Mar. 5, 2026

(54) ELECTROMYOGRAPHY STRAP AND WEARABLE ELECTRONIC DEVICE

(71) Applicant: Vitaly Medical, Inc., Los Angeles, CA (US)

(72) Inventors: Xiaoyang Yu, Los Angeles, CA (US); Suoyi Feng, Berkeley, CA (US); Liheng Lai, Berkeley, CA (US); Yan Li, Fort Lee, NJ (US)

(21) Appl. No.: 19/264,811

(22) Filed: Jul. 9, 2025

Related U.S. Application Data
(60) Provisional application No. 63/687,407, filed on Aug. 27, 2024.

(51) Int. Cl. A61B 5/256; A61B 5/00; A61B 5/263; A61B 5/296; A61B 5/313

(52) U.S. Cl. CPC A61B 5/256; A61B 5/263; A61B 5/296; A61B 5/313; A61B 5/681; A61B 2560/0443; A61B 2562/043; A61B 2562/164; A61B 2562/166; A61B 2562/227

(57) ABSTRACT

An electromyography (EMG) strap and a wearable electronic device are provided. The EMG strap includes a flexible strap body; a flexible printed circuit board (FPCB), housed within the flexible strap body; a surface electromyography (sEMG) sensor assembly, disposed on the FPCB, where the sEMG sensor assembly is exposed from the flexible strap body and is configured to collect an EMG signal and pre-process the EMG signal to obtain a pre-processed analog signal; and a board-to-board (BTB) connector, disposed on the FPCB and electrically connected to the sEMG sensor assembly. The EMG strap can not only help in diagnosing conditions like muscular dystrophy and motor neuron disease but also play a pivotal role in developing advanced prosthetic devices that respond to muscle signals.

### TECHNICAL FIELD

The present disclosure relates to the field of electromyography (EMG) sensing technologies, and in particularly, to an EMG strap and a wearable electronic device.

### DESCRIPTION OF RELATED ART

Electronic devices are commonplace throughout most of the world today. Advancements in integrated circuit technology have enabled the development of electronic devices that are sufficiently small and lightweight to be carried by the user. Such “portable” electronic devices may include on-board power supplies (such as batteries or other power storage systems) and may be designed to operate without any wire-connections to other electronic systems. However, a small and lightweight electronic device may still be considered portable even if it includes a wire-connection to another electronic system. For example, a microphone may be considered a portable electronic device whether it is operated wirelessly or through a wire-connection.

The convenience afforded by the portability of electronic devices has fostered a huge industry. Smart phones, audio players, laptop computers, tablet computers, and eBooks readers are all examples of portable electronic devices. However, the convenience of being able to carry a portable electronic device has also introduced the inconvenience of having one's hand(s) encumbered by the device itself. This problem is addressed by making an electronic device not only portable, but wearable.

A wearable electronic device is any portable electronic device that a user can carry without physically grasping, clutching, or otherwise holding onto the device with their hands. For example, a wearable electronic device may be attached or coupled to the user by a strap or straps, a band or bands, a clip or clips, an adhesive, a pin and clasp, an article of clothing, tension or elastic support, an interference fit, an ergonomic form, etc. Examples of wearable electronic devices include digital wristwatches, electronic armbands, electronic rings, electronic ankle-bracelets or “anklets”, head-mounted electronic display units, hearing aids, and so on.

A wearable electronic device may provide direct functionality for a user (such as audio playback, data display computing functions, etc.) or it may provide electronics to interact with, receive information from, or control another electronic device. For example, a wearable electronic device may include sensors that detect inputs affected by a user and transmit signals to another electronic device based on those inputs. Sensor-types and input-types may each take on a variety of forms, including but not limited to: tactile sensors (e.g. buttons, switches, touchpads, or keys) providing manual control, acoustic sensors providing voice-control, electromyography sensors providing gesture control, and/or accelerometers providing gesture control.

A human-computer interface (“HCI”) is an example of a human-electronics interface. The present systems, articles, and methods may be applied to HCIs, but may also be applied to any other form of human-electronics interface.

The concept of a wristwatch with on-board computation capabilities and functionality beyond timekeeping (i.e., a “smart watch”) has been around for decades. Seiko and Casio were building digital wristwatches with user-programmable memory and computing capability as far back as in the 1980s. However, at least because of their limited functionality, the initial designs for smartwatches never took off in consumer markets.

Motivated by the availability of more advanced integrated circuit, display, and battery technologies, there has recently been a resurgence in the smartwatch industry. Exemplary smart watches that are currently known to be under development include: an Apple Watch™, the Samsung Galaxy Gear™, the Sony Smart Watch™, the Qualcomm Toq™, and the Pebble™ by Pebble Technology. Each of these examples provides (or is expected to provide) various functions and capabilities and employs a unique design and geometry. However, all these designs are fundamentally similar in that they essentially emulate the design of a traditional wristwatch. That is, each design includes a housing that is physically coupled to a strap or band that fits around the user's wrist, the housing having a display on one side and a back-plate proximate the user's wrist on the side opposite the display. Conforming to this generic arrangement is a design constraint for virtually any smart watch, as most smart watches are designed to resemble the traditional wristwatch as much as possible.

The back-plate that is common to all known wristwatch designs (both traditional and smart watches alike) provides structural support and protects the internal com-ponents (circuitry or gears, etc.) of the wristwatch from its environment. Otherwise, the back-plate that is common to all known wrist watch designs does not typically provide or enable other functions and/or capabilities of the wristwatch. Similarly, the strap or band (or similar, hereafter “watch-strap”) that is common to virtually all known wristwatch designs (both traditional and smart watches alike) typically serve one purpose: holding the watch in position on the user's wrist. Beyond this, the watch strap that is common to virtually all known wrist watch designs do not typically import or enable any functionality or capability in the watch itself.

Electromyography (“EMG”) is a process for detecting and processing the electrical signals generated by muscle activity, EMG devices employ EMG sensors that are responsive to the range of electrical potentials (typically uV-mV involved in muscle activity, EMG signals may be used in a wide variety of applications, including: medical monitoring and diagnosis, muscle rehabilitation, exercise and training, prosthetic control, and even controlling functions electronic devices (i.e., in human-electronics interfaces).

There are two main types of EMG sensors: intramuscular EMG sensors and surface EMG sensors. As the name suggest, intramuscular EMG sensors are designed to penetrate the skin and measure EMG signals from within the muscle tissue, while surface EMG sensors are designed to rest on an exposed surface of the skin and measure EMG signals from there. Intramuscular EMG sensor measurements can be much more precise than surface EMG sensor measurements: however, intramuscular EMG sensors must be applied by a trained professional, are obviously more invasive, and are less desirable from the patient's point of view, The use of intramuscular EMG sensors is generally limited to clinical settings.

Surface EMG sensors can be applied with ease, are much more comfortable for the patient/user, and are therefore more appropriate for non-clinical settings and uses. For example, human-electronics interfaces that employ EMG, such as those proposed in U.S. patents with publication U.S. Pat. Nos. 6,244,873B1 and 8,170,656B2, usually employ surface EMG sensors. Surface EMG sensors are available in two forms: resistive EMG sensors and capacitive EMG sensors. The electrode of a resistive EMG sensor is typically directly electrically coupled to the user's skin while the electrode of a capacitive EMG sensor is typically capacitively coupled to the user's skin. That is, for a resistive EMG sensor, the electrode typically includes a plate of electrically conductive material that is in direct physical contact with the user's skin. While for a capacitive EMG sensor, the electrode typically includes a plate of electrically conductive material that is electrically insulated from the user's skin by at least one thin intervening layer of dielectric material or cloth.

Resistive EMG sensors and capacitive EMG sensors both have relative advantages and disadvantages. For example, the resistive coupling to the skin realized by a resistive EMG sensor provides a relatively low impedance (compared to a capacitive coupling) between the skin and the sensor and this can greatly simplify the circuitry needed to amplify the detected EMG signals; however, because this resistive coupling is essentially galvanic and uninterrupted, it can also undesirable couple DC voltage to the amplification circuitry and/or result in a voltage applied to the skin of the user. Both of these effects potentially impact the quality of the EMG signals detected. On the other hand, the capacitive coupling to the skin realized by a capacitive EMG sensor galvanically isolated the amplification circuitry from the skin and thereby prevents a DC voltage from coupling to the amplification circuitry and prevents a voltage from being applied to the skin. However, this capacitive coupling provides a relatively high impedance between the skin and the sensor and this can complicate the circuitry needed to amplify the detected EMG signals (thus making the amplification circuitry more expensive). The strength of the capacitive coupling can also vary widely from user to user. Apparently, neither of the resistive EMG sensor and the capacitive EMG sensor is ideal and there is a need in the art for improved surface EMG sensor designs.

Further, Electromyography (EMG) sensors have become invaluable tools in the field of biomedical engineering, particularly for the diagnosis and monitoring of neuromuscular conditions. By capturing the electrical activity produced by skeletal muscles, EMG sensors provide crucial insights into muscle function, enabling clinicians and researchers to assess muscle health, detect abnormalities, and guide rehabilitation. The ability to measure muscle activity in real-time offers significant advantages, especially in fields like physical therapy, sports science, and neuroprosthetics systems. EMG sensors not only help in diagnosing conditions like muscular dystrophy and motor neuron disease but also play a pivotal role in developing advanced prosthetic devices that respond to muscle signals.

For many clinical and research applications, there is a growing need for long-term, continuous monitoring of muscle activity. Such continuous monitoring is essential for accurately tracking changes in muscle function over time, particularly in patients undergoing rehabilitation or those with chronic neuromuscular disorders. Traditional EMG monitoring systems, which often rely on bulky, wired equipment, are not suited for long-term use, as they can cause discomfort and inconvenience to the wearer. To address this need, integrating EMG sensors into wearable devices that can be comfortably worn for extended periods is crucial. These wearable devices should be designed to be lightweight, unobtrusive, and capable of providing high-quality, continuous data without interrupting the user's daily activities.

Traditional EMG sensors, which are typically rigid and require direct contact with the skin, often face issues with maintaining consistent contact with the tested muscles. This can lead to unreliable readings and discomfort for the user, particularly during long-term monitoring.

### SUMMARY

An objective of the present disclosure is to provide an EMG strap and a wearable electronic device, which can resolve the technical problems in the related art that the traditional rigid EMG sensors cannot ensure consistent contact with the test muscles, thereby leading to unreliable readings and discomfort for the user, particularly during long-term monitoring.

In a first aspect, an embodiment of the present disclosure provides an EMG strap. The EMG strap includes: a flexible strap body; a flexible printed circuit board (FPCB), housed within the flexible strap body; a surface electromyography (sEMG) sensor assembly, disposed on the FPCB, the sEMG sensor assembly is exposed from the flexible strap body and is configured to collect an EMG signal and pre-process the EMG signal to obtain a pre-processed analog signal; and a board-to-board (BTB) connector, disposed on the FPCB and electrically connected to the sEMG sensor assembly.

In an embodiment, the flexible strap body is two in number, and the two flexible strap bodies are fixed connected or detachably connected; the FPCB is two in number, the two FPCBs are respectively housed within the two flexible strap bodies; the sEMG sensor assembly is two in number; and the two sEMG sensor assemblies are respectively disposed on the two FPCBs and respectively exposed from the two flexible strap bodies; and the BTB connector is two in number, and the two BTB connectors are respectively disposed on the two FPCBs and respectively electrically connected to the two sEMG sensor assemblies.

In an embodiment, the two BTB connectors are exposed from first ends of the two flexible strap bodies, respectively; and second ends of the two flexible strap bodies are detachably connected to each other.

In an embodiment, each of the two sEMG sensor assemblies includes an analog front-end (AFE) circuit, disposed on a first surface of a corresponding one of the two FPCBs and electrically connected to a corresponding one of the two BTB connectors; and an electrode set, disposed on a second surface of the corresponding one of the two FPCBs and electrically connected to the AFE circuit, where the electrode set is exposed from a corresponding one of the two flexible strap bodies.

In an embodiment, the AFE circuit includes a preamplifier, a twin-T notch filter, a high-pass filter, a low-pass filter, and a variable gain buffer, which are sequentially electrically connected in that order; the preamplifier is electrically connected between the electrode set and the twin-T notch filter; and the variable gain buffer is electrically connected to the corresponding one of the two BTB connectors.

In an embodiment, the electrode set includes a first differential electrode, a reference electrode, and a second differential electrode; and the first differential electrode, the reference electrode, and the second differential electrode are electrically connected to the preamplifier.

In an embodiment, the preamplifier is an instrumentation amplifier.

In an embodiment, each of the two flexible strap bodies includes an upper shell and a lower shell, the upper shell and the lower shell together define a accommodation cavity, and a corresponding one of the two FPCBs is housed in the accommodation cavity; and the lower shell defines an opening, and the electrode set is exposed from the lower shell via the opening.

In an embodiment, the AFE circuit in each of the two sEMG sensor assemblies is two in number, the electrode set in each of the two sEMG sensor assemblies is two in number, the opening includes two groups of openings, and each group of the two groups of openings consists of three gaps separated from each other.

In an embodiment, each of the first differential electrode, the reference electrode, and the second differential electrode is made from copper-plated nickel.

In an embodiment, a size of each of the first differential electrode, the reference electrode, and the second differential electrode is 5 mm×3 mm.

In an embodiment, a spacing between the two electrode sets is 3.5 cm.

In an embodiment, each of the upper shell and the lower shell is made from a flexible silicone material.

In an embodiment, each of the first differential electrode, the reference electrode, and the second differential electrode is a dry electrode.

In a second aspect, an embodiment of the present disclosure provides a wearable electronic device, which includes the EMG strap described above and a device body. The device body includes a processor and is mechanically and electrically connected to the EMG strap. The processor is electrically connected to the BTB connector and is configured to receive and process the pre-processed analog signal to obtain a digital signal.

In an embodiment, the processor is a micro-processing unit (MPU), the MPU includes an analog-to-digital converter (ADC) and a storage unit, the ADC is configured to convert the pre-processed analog signal into the digital signal, the storage unit is configured to store the digital signal, and the BTB connector is configured to transmit the pre-processed analog signal to the MPU.

In an embodiment, the device body further includes a battery, electrically connected to the processor and configured to provide power for the processor and the FPCB.

In a third aspect, another EMG strap is provided, which includes: two flexible silicone shells, respectively defining two accommodation cavities; two FPCBs, respectively housed within the two accommodation cavities; four AFE circuits, two of the four AFE circuits are disposed on one of the two FPCBs, and another two of the four AFE circuits are disposed on another of the two FPCBs; four electrode sets, two of the four electrode sets are disposed on the one of the two FPCBs and respectively electrically connected to the two of the four AFE circuits, and are exposed from one of the two flexible silicone shells; and another two of the four electrode sets are disposed on the another of the two FPCBs and respectively electrically connected to the another two of the four AFE circuits, and are exposed from another of the two flexible silicone shells; and two BTB connectors, respectively disposed on the two FPCBs, the two BTB connectors are respectively exposed from two first ends of the two flexible silicone shells, one of the two BTB connectors is electrically connected to the two of the four AFE circuits disposed on the one of the two FPCBs, another of the two BTB connectors is electrically connected to the another two of the four AFE circuits disposed on another of the two FPCBs, and two second ends of the two flexible silicone shells are detachably connected to each other.

In an embodiment, each of the four AFE circuits includes a preamplifier, a twin-T notch filter, a high-pass filter, a low-pass filter, and a variable gain buffer, which are sequentially electrically connected in that order; the preamplifier is electrically connected between a corresponding one of the four electrode sets and the twin-T notch filter; and the variable gain buffer is electrically connected to a corresponding one of the two BTB connectors.

In an embodiment, each of the two flexible silicone shells defines openings for exposing corresponding two of the four electrode sets.

The present disclosure may have at least one of the following beneficial effects.

In the EMG strap of the present disclosure, the EMG strap includes the flexible strap body, the FPCB, the sEMG sensor assembly, and the BTB connector. The FPCB is housed within the flexible strap body. The sEMG sensor assembly is disposed on the FPCB, is exposed from the flexible strap body, and is configured to collect an EMG signal and pre-process the EMG signal to obtain a pre-processed analog signal. The BTB connector is disposed on the FPCB and electrically connected to the sEMG sensor assembly. Owing to the flexible strap body and the FPCB, the EMG strap is flexible, the EMG strap can be easily adjusted to ensure close, secure contact with a target muscle. The flexibility of the EMG strap allows it to conform to a natural contour of a to-be-tested body, reducing the risk of contact issues and improving the overall accuracy and reliability of the measurements. By addressing the limitations of traditional EMG sensors, the EMG strap enhances the practicality and effectiveness of long-term EMG monitoring in wearable medical devices.

Moreover, the EMG strap exhibits structural adaptability that enables it to function as a band component for various types of wearable electronic devices. It can be integrated into existing wearable formats, including but not limited to wristbands, watch straps, armbands, thigh straps, calf straps, and ankle bands, without necessitating substantial modifications to the device housing or overall form factor. This configuration allows for seamless incorporation of surface electromyographic (sEMG) sensing functionality into commercially available wearable products, such as smartwatches or fitness trackers. The EMG strap is suitable for placement at various anatomical locations, including the wrist, arm, thigh, calf, or ankle, thereby supporting flexible deployment across different body regions. As such, the EMG strap enables continuous acquisition of neuromuscular activity signals while facilitating unobtrusive, long-term health monitoring in everyday use scenarios.

### BRIEF DESCRIPTION OF THE DRAWINGS

In order to explain embodiments of the present disclosure more clearly, accompanying drawings that need to be used in the description of the embodiments are briefly introduced hereinafter. It is apparent that the accompanying drawings in the following description are merely some embodiments of the present disclosure, and for the skilled in the art, other drawings can also be obtained according to the structures shown in these introduced drawings without creative efforts.

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as a non-limiting illustrative example.

FIG. 1 illustrates a schematic perspective view of a wearable electronic device according to an embodiment of the present disclosure.

FIG. 2 illustrates a schematic disassembled view of the wearable electronic device in FIG. 1, in which an outer side of the wearable electronic device is shown.

FIG. 3 illustrates another schematic disassembled view of the wearable electronic device in FIG. 1, in which an inner side of the wearable electronic device is shown.

FIG. 4 illustrates a schematic exploded view of an EMG strap of the wearable electronic device in FIG. 1.

FIG. 5 illustrates a schematic structural view of AFE circuits and electrode sets on a FPCB of the EMG strap in FIG. 4, in which, (a) shows a first surface of the FPCB, and (b) shows a second surface of the FPCB.

FIG. 6 illustrates a schematic connection view of an AFE circuit and an electrode set of the EMG strap in FIG. 4.

FIG. 7 illustrates a schematic structure diagram of a device body of the wearable electronic device in FIG. 1.

FIG. 8 illustrates a frequency response of a twin-T notch filter of an AFE circuit of the EMG strap in FIG. 4, showing a sharp attenuation of −40 dB around 60 Hz and 0 dB for other frequencies.

FIG. 9 illustrates a frequency response of a high-pass filter of the AFE circuit of the EMG strap in FIG. 4, showing a cutoff frequency at 15 Hz with a gain of 20 dB.

FIG. 10 illustrates a frequency response of a low-pass filter of the AFE circuit of the EMG strap in FIG. 4, with a cutoff frequency at 500 Hz and unity gain.

FIG. 11 illustrates a frequency response from 0 Hz to 1000 Hz, revealing an overall gain of 60 dB.

### DESCRIPTION OF REFERENCE NUMERALS

100—wearable electronic device; 10—EMG strap; 12—flexible strap body (flexible silicone shell); 120—first end; 122—second end; 124—upper shell; 126—lower shell; 1260—opening (gap); 128—accommodation cavity; 14—FPCB; 142—first surface; 144—second surface; 16—sEMG sensor assembly; 162—AFE circuit; 1620—Preamplifier; 1624—twin-T notch filter; 1626—high-pass filter; 1628—low-pass filter; 1630—variable gain buffer; 164—electrode set; 1640—first differential electrode; 1642—reference electrode; 1644—second differential electrode; 18—BTB connector; 20—device body; 22—processor (MPU); 220—ADC; 222—storage unit; 24—battery.

### DETAILED DESCRIPTION OF EMBODIMENTS

Technical solutions in embodiments of the present disclosure will be clearly and completely described below combined with the accompanying drawings in the embodiments of the present disclosure. It is apparent that the described embodiments are merely part of embodiments of the present disclosure, not all of the embodiments of the embodiments. Based on the described embodiments of the present disclosure, all other embodiments obtained by the skilled in the art without creative efforts shall fall within the scope of protection of the present disclosure.

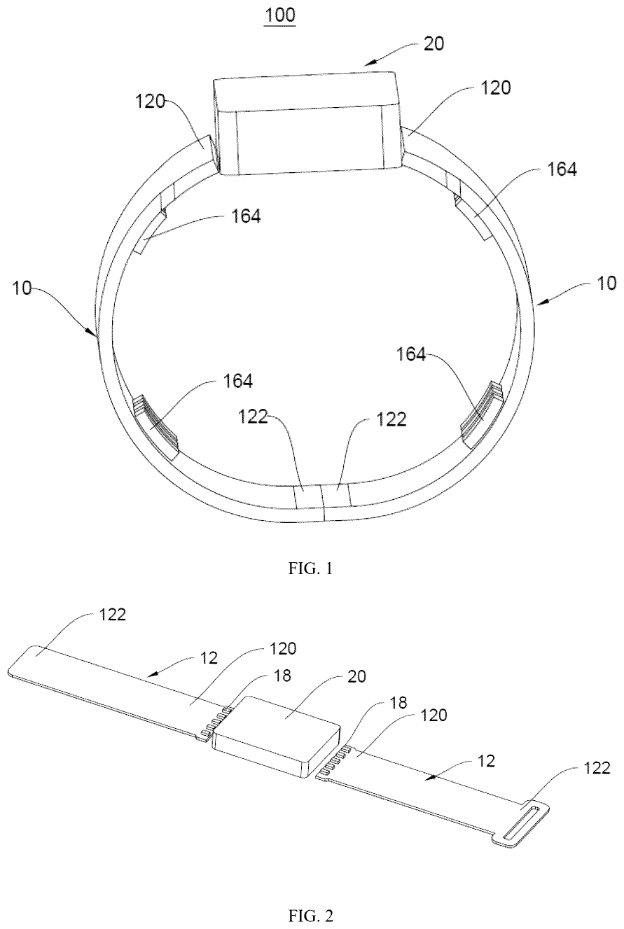

Referring to FIG. 1, a wearable electronic device 100 is provided, which can be worn around various limbs, such as wrists, arms, legs, or ankles. The wearable electronic device 100 includes two EMG straps 10 and a device body 20 mechanically and electrically connected between the two EMG straps 10. Two ends of the two EMG straps 10 facing away from the device body 20 are fixed connected or detachably connected.

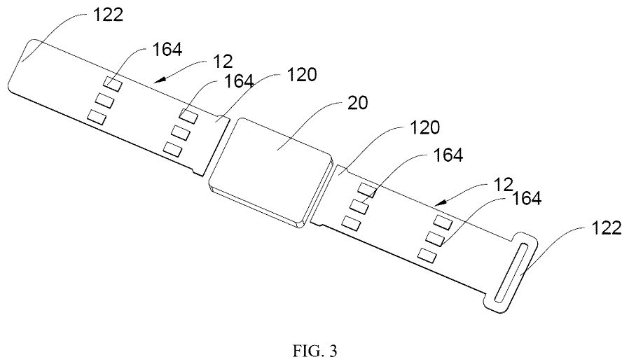

As shown in FIG. 2 through FIG. 5, each of the two EMG straps 10 includes a flexible strap body (also referred to as flexible shell) 12, an FPCB 14, an sEMG sensor assembly 16, and a BTB connector 18. The FPCB 14 is housed within the flexible strap body 12. The sEMG sensor assembly 16 is disposed on the FPCB 14. The sEMG sensor assembly 16 is exposed from the flexible strap body 12 and is configured to collect an EMG signal and pre-process the EMG signal to obtain a pre-processed analog signal. The BTB connector 18 is disposed on the FPCB 14 and electrically connected to the sEMG sensor assembly 16. Owing to the flexible strap body 12 and the FPCB 14 for providing high flexibility and lightweight characteristics, the EMG strap 10 is flexible, the wearable electronic device 100 with the EMG strap 10 can be easily adjusted to ensure close and secure contact with a target muscle. The flexibility of each EMG strap 10 allows it to conform to a natural contour of a to-be-tested body, reducing the risk of contact issues and improving the overall accuracy and reliability of the measurements. By addressing the limitations of traditional EMG sensors, the EMG strap 10 enhances the practicality and effectiveness of long-term EMG monitoring in the wearable electronic device 100.

As shown in FIG. 2, in an embodiment, in the wearable electronic device 100, the two BTB connectors 18 are exposed from first ends 120 of the two flexible strap bodies 12, respectively. Second ends 122 of the two flexible strap bodies 12 are detachably connected to each other. For example, the second end 122 of one of the two flexible strap bodies 12 is provided with a hole, and the second end 122 of the other of the two flexible strap bodies 12 is configured to pass through the hole to allow to adjust a size of a space defined by the two EMG straps 10 and the device body 20 for matching different limb sizes, thereby facilitating proper contact and ensuring a comfortable wearing experience. The wired communication between the two sEMG sensor assemblies 16 in the two EMG straps 10 is achieved using the two BTB connectors 220, which helps avoid data asynchrony issues that may arise with wireless connections.

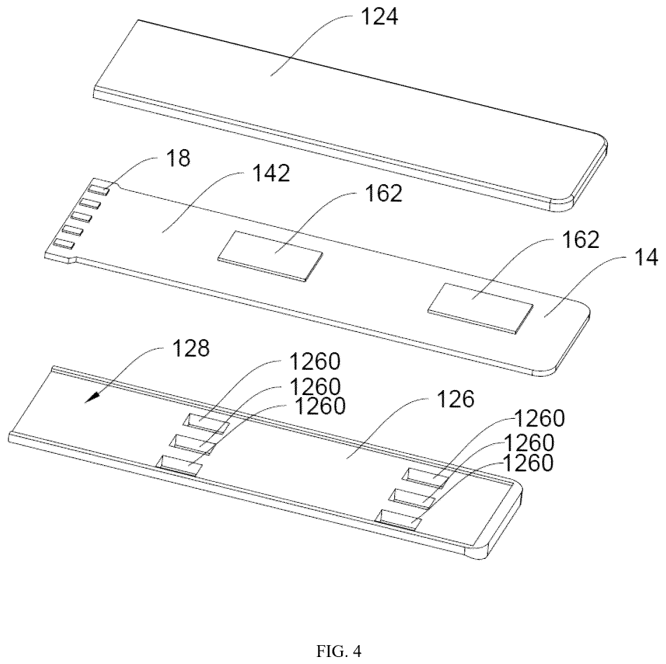

As shown in FIG. 4, in an embodiment, the flexible strap body 12 includes an upper shell 124 and a lower shell 126. The upper shell 124 and the lower shell 126 together define an accommodation cavity 128. The FPCB 14 is housed within the accommodation cavity 128. The lower shell 126 defines an opening 1260, and the sEMG sensor assembly 16 is exposed from the lower shell 126 via the housing 1260. For example, each of the upper shell 124 and the lower shell 126 is made from a flexible silicone material, silicone's physical properties provide excellent skin adhesion while ensuring comfort during wear, a lower electrical conductivity of the flexible silicone material also protects the circuit therein. The lower shell 126 also defines a groove designed to house the FPCB 14, which reduces internal stress and provides additional protection for the FPCB 14 and the sEMG sensor assembly 16 thereon.

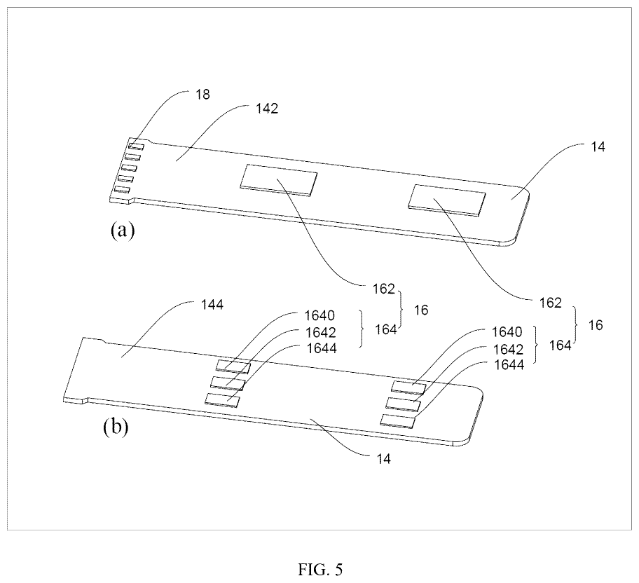

As shown in FIG. 4 and FIG. 5, in an embodiment, the sEMG sensor assembly 16 includes an AFE circuit 162 and an electrode set 164. The AFE circuit 162 is disposed on a first surface 142 of the FPCB 14 and electrically connected to the BTB connector 18. The electrode set 164 is disposed on a second surface 144 of the FPCB 14 and electrically connected to the AFE circuit 162. The electrode set 164 is exposed from the opening 1260 on the lower shell 126. When a user applies the EMG strap 10, it is essential to maintain constant contact between the electrode set 164 and a corresponding limb to ensure continuous collection of electrical signals.

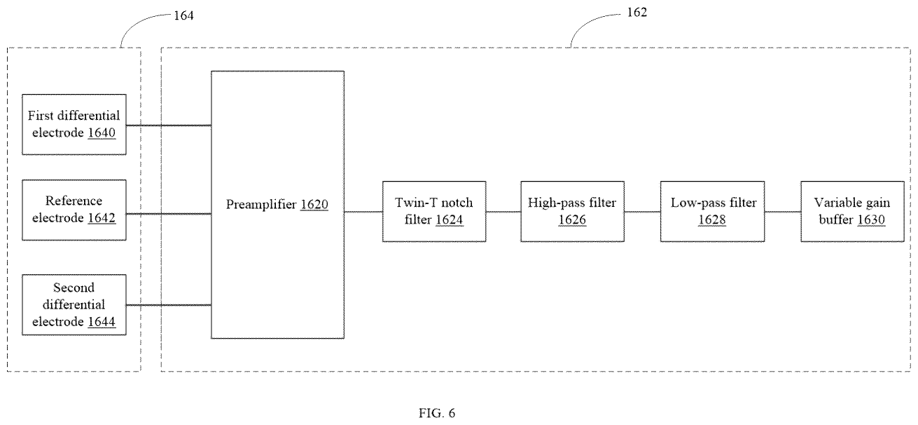

As shown in FIG. 6, in an embodiment, the AFE circuit 162 includes: a preamplifier 1620, a twin-T notch filter 1624, a high-pass filter 1626, a low-pass filter 1628, and a variable gain buffer 1630, which are sequentially electrically connected in that order. The preamplifier 1620 is electrically connected between the electrode set 164 and the twin-T notch filter 1624. The variable gain buffer 1630 is electrically connected to the BTB connector 18. This design of the AFE circuit 162 ensures a high signal-to-noise ratio (SNR) and sufficient signal strength for subsequent digitization and digital processing.

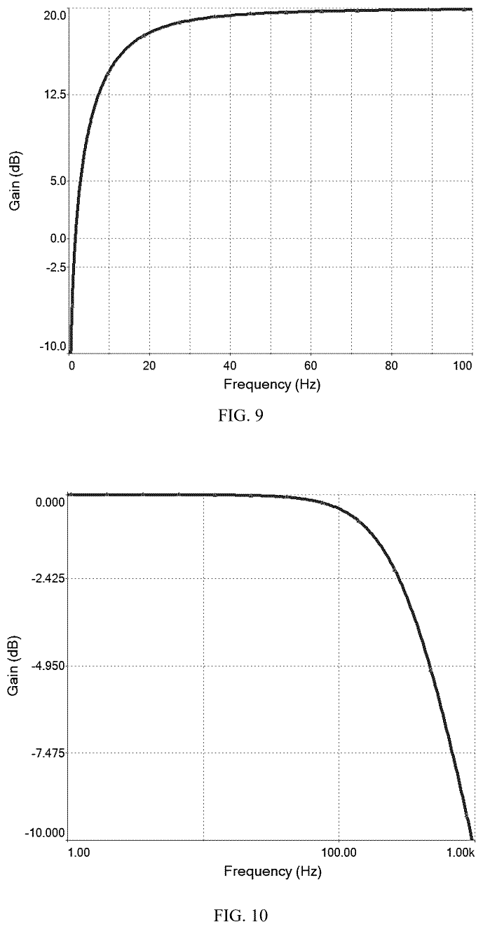

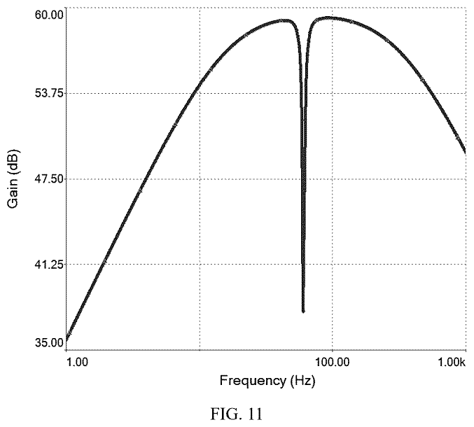

As shown in FIG. 6, in an embodiment, the electrode set 164 includes a first differential electrode 1640, a reference electrode 1642, and a second differential electrode 1644. For example, the preamplifier 1620 is an instrumentation amplifier and is configured to obtain a differential signal. For example, the twin-T notch filter 1624 has a notch frequency of 60 Hz or 50 Hz (the notch frequency depends on a region) and is configured to remove significant electromagnetic interference (EMI) caused by unavoidable power frequencies in daily environments. The high-pass filter 1626 has a cutoff frequency of 15 Hz and is configured to eliminate low-frequency motion artifacts. The low-pass filter 1628 has a cutoff frequency of 500 Hz and is configured to discard frequencies outside the EMG signal bandwidth. The variable gain buffer 1630 is configured to adjust a signal amplitude based on muscle type, ensuring the EMG signal is within a suitable voltage range for digitization and further processing. Referring to FIG. 8, FIG. 9, FIG. 10 and FIG. 11, bode diagrams of the EMG circuit demonstrate the superior performance of the AFE circuit 162. It should be noted, a specific structure of each of the preamplifier 1620, the twin-T notch filter 1624, the high-pass filter 1626, the low-pass filter 1628, and the variable gain buffer 1630 is not limited herein, as long as the above functions thereof can be achieved.

Further, each of the first differential electrode 1640, the reference electrode 1642, and the second differential electrode 1644 is made from copper-plated nickel, thereby ensuring excellent electrical conductivity and strong skin adhesion. A size of each of the first differential electrode 1640, the reference electrode 1642, and the second differential electrode 1644 is 5 mm×3 mm. When the wearable electronic device 100 works, the EMG signal is collected through the first differential electrode 1640, the reference electrode 1642, and the second differential electrode 1644. These electrodes serve different functions: the first differential electrode 1640 is a positive input, the reference electrode 1642 is a reference input, and second differential electrode 1644 is a negative input for the EMG signal.

Further, in the present disclosure, the first differential electrode 1640, the reference electrode 1642, and the second differential electrode 1644 are dry electrodes, rather than traditional wet electrodes (e.g., hydrogel-based electrodes). Compared to wet electrodes, the dry electrodes offer the advantages of reusability, simplified preparation, and reduced skin irritation. Additionally, their surface conductivity can be further improved by applying conductive gel when necessary, ensuring both flexibility in usage and reliable biopotential signal acquisition.

Moreover, the electrode set 164 on a single FPCB 14 is at least one in number. For example, in FIG. 4, there are two electrode sets 164 on the single FPCB 14. Of course, the present disclosure does not limit the number of the electrode set 164 on the single FPCB 14. In a situation that there are at least two electrode sets on the single FPCB 14, a spacing between two adjacent electrode sets is 3.5 cm. Also, the AFE circuit 162 on the single FPCB 14 is also at least one in number, and the number of the AFE circuit 162 on the single FPCB 14 is equal to the number of the electrode set 164 on the single FPCB 14. When there are two electrode sets 164 and two AFE circuits 162 on the single FPCB 14, which ensures accurate EMG measurements, two electrode sets 164 are electrically connected to the two AFE circuits 162, respectively, the lower shell 126 defines two groups of openings for exposing the two electrode sets 164 from the lower shell 126, and each group of the two groups of openings consists of three gaps 1260 separated from each other. The gaps 1260 ensure effective contact between the electrode sets 164 and a skin of a tested object for accurate EMG signal acquisition.

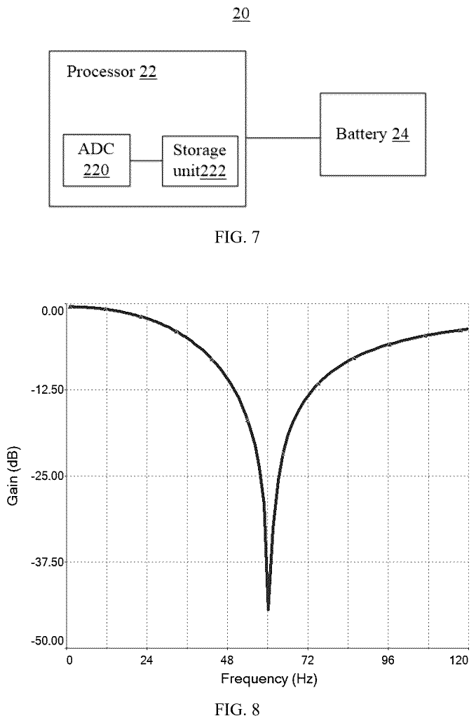

As shown in FIG. 7, in an embodiment, the device body 20 includes a processor 22. The processor 22 is electrically connected to the two BTB connectors 18 of the two EMG straps 10 and is configured to receive and process the pre-processed analog signal from each of the two EMG straps 10 to obtain a digital signal. For example, the processor 22 is an MPU, as a core processing unit of the wearable electronic device 100. The MPU includes an ADC 220 and a storage unit 222. The ADC 220 is configured to convert the pre-processed analog signal into the digital signal, and the ADC 220 has a sampling frequency higher than 1000 Hz to satisfy the Nyquist criterion and prevent aliasing for EMG signals within a bandwidth of 20 Hz to 500 Hz. In practice, a sampling rate of the ADC 220 is typically set above 1000 Hz to allow for more accurate signal reconstruction and digital filtering. The ADC 220 has a resolution higher than 12 bits to maintain higher data quality. The storage unit 222 is configured to store the digital signal. The storage unit 222 has a sufficient capacity to accommodate the higher sampling frequency, ensuring that the digitized EMG signals are stored for further analysis. Each BTB connector 18 is configured to transmit the pre-processed analog signal to the MPU. Further, the device body 20 further includes a battery 24 and a power management unit, which is electrically connected to the processor 22 and configured to provide power for the processor 22 and the FPCB 14. Moreover, the MPU is equipped with essential components for processing the pre-processed analog signal from the EMG strap 10.

Further, even though a specific structure of the device body 20 is described above, it should be noted that, the present disclosure does not limit the specific structure of the device body 20, as long as the device body can achieve the corresponding function.

In summary, the present disclosure provides the EMG strap 10 and the wearable electronic device 100 containing the EMG strap 10. The EMG strap 10 includes the flexible strap body 12, the FPCB 14, the sEMG sensor assembly 16, and the BTB connector 18. The FPCB 14 is housed within the flexible strap body 12. The sEMG sensor assembly 16 is disposed on the FPCB 14, is exposed from the flexible strap body 12, and is configured to collect an EMG signal and pre-process the EMG signal to obtain a pre-processed analog signal. The BTB connector 18 is disposed on the FPCB 14 and electrically connected to the sEMG sensor assembly 16. Owing to the flexible strap body 12 and the FPCB 14, the EMG strap 10 is flexible, the EMG strap 10 can be easily adjusted to ensure close, secure contact with a target muscle. The flexibility of the EMG strap 10 allows it to conform to a natural contour of a to-be-tested body, reducing the risk of contact issues and improving the overall accuracy and reliability of the measurements. By addressing the limitations of traditional EMG sensors, the EMG strap 10 enhances the practicality and effectiveness of long-term EMG monitoring in the wearable electronic device 100.

Moreover, the EMG strap 10 exhibits structural adaptability that enables it to function as a band component for various types of wearable electronic devices. It can be integrated into existing wearable formats, including but not limited to wristbands, watch straps, armbands, thigh straps, calf straps, and ankle bands, without necessitating substantial modifications to the device housing or overall form factor. This configuration allows for seamless incorporation of surface electromyographic (sEMG) sensing functionality into commercially available wearable products, such as smartwatches or fitness trackers. The EMG strap 10 is suitable for placement at various anatomical locations, including the wrist, arm, thigh, calf, or ankle, thereby supporting flexible deployment across different body regions. As such, the EMG strap enables continuous acquisition of neuromuscular activity signals while facilitating unobtrusive, long-term health monitoring in everyday use scenarios.

Although the present disclosure provides certain example embodiments and applications, other embodiments that are apparent to those of ordinary skill in the art, including embodiments which do not provide all of the features and advantages set forth herein, are also within the scope of the present disclosure.

What is claimed is:

1. An electromyography (EMG) strap, comprising:
a flexible strap body;
a flexible printed circuit board (FPCB), housed within the flexible strap body;
a surface electromyography (sEMG) sensor assembly, disposed on the FPCB, wherein the sEMG sensor assembly is exposed from the flexible strap body and is configured to collect an EMG signal and pre-process the EMG signal to obtain a pre-processed analog signal; and
a board-to-board (BTB) connector, disposed on the FPCB and electrically connected to the sEMG sensor assembly.

2. The EMG strap as claimed in claim 1, wherein the flexible strap body is two in number, and the two flexible strap bodies are fixed connected or detachably connected;
wherein the FPCB is two in number, the two FPCBs are respectively housed within the two flexible strap bodies;
wherein the sEMG sensor assembly is two in number; and the two sEMG sensor assemblies are respectively disposed on the two FPCBs and respectively exposed from the two flexible strap bodies; and
wherein the BTB connector is two in number, and the two BTB connectors are respectively disposed on the two FPCBs and respectively electrically connected to the two sEMG sensor assemblies.

3. The EMG strap as claimed in claim 2, wherein the two BTB connectors are exposed from first ends of the two flexible strap bodies, respectively; and second ends of the two flexible strap bodies are detachably connected to each other.

4. The EMG strap as claimed in claim 2, wherein each of the two sEMG sensor assemblies comprises:
an analog front-end (AFE) circuit, disposed on a first surface of a corresponding one of the two FPCBs and electrically connected to a corresponding one of the two BTB connectors; and
an electrode set, disposed on a second surface of the corresponding one of the two FPCBs and electrically connected to the AFE circuit, wherein the electrode set is exposed from a corresponding one of the two flexible strap bodies.

5. The EMG strap as claimed in claim 4, wherein the AFE circuit comprises: a preamplifier, a twin-T notch filter, a high-pass filter, a low-pass filter, and a variable gain buffer, which are sequentially electrically connected in that order; the preamplifier is electrically connected between the electrode set and the twin-T notch filter; and the variable gain buffer is electrically connected to the corresponding one of the two BTB connectors.

6. The EMG strap as claimed in claim 5, wherein the electrode set comprises a first differential electrode, a reference electrode, and a second differential electrode; and
wherein the first differential electrode, the reference electrode, and the second differential electrode are each electrically connected to the preamplifier.

7. The EMG strap as claimed in claim 6, wherein the preamplifier is an instrumentation amplifier.

8. The EMG strap as claimed in claim 6, wherein each of the two flexible strap bodies comprises an upper shell and a lower shell, the upper shell and the lower shell together define a accommodation cavity, and a corresponding one of the two FPCBs is housed in the accommodation cavity; and
wherein the lower shell defines an opening, and the electrode set is exposed from the lower shell via the opening.

9. The EMG strap as claimed in claim 8, wherein the AFE circuit in each of the two sEMG sensor assemblies is two in number, the electrode set in each of the two sEMG sensor assemblies is two in number, the opening comprises two groups of openings, and each group of the two groups of openings consists of three gaps separated from each other.

10. The EMG strap as claimed in claim 6, wherein each of the first differential electrode, the reference electrode, and the second differential electrode is made from copper-plated nickel.

11. The EMG strap as claimed in claim 6, wherein a size of each of the first differential electrode, the reference electrode, and the second differential electrode is 5 mm×3 mm.

12. The EMG strap as claimed in claim 9, wherein a spacing between the two electrode sets is 3.5 cm.

13. The EMG strap as claimed in claim 8, wherein each of the upper shell and the lower shell is made from a flexible silicone material.

14. The EMG strap as claimed in claim 7, wherein each of the first differential electrode, the reference electrode, and the second differential electrode is a dry electrode.

15. A wearable electronic device, comprising:
the EMG strap as claimed in claim 1; and
a device body, comprising a processor and mechanically and electrically connected to the EMG strap, wherein the processor is electrically connected to the BTB connector and is configured to receive and process the pre-processed analog signal to obtain a digital signal.

16. The wearable electronic device as claimed in claim 15, wherein the processor is a micro-processing unit (MPU), the MPU comprises an analog-to-digital converter (ADC) and a storage unit, the ADC is configured to convert the pre-processed analog signal into the digital signal, the storage unit is configured to store the digital signal, and the BTB connector is configured to transmit the pre-processed analog signal to the MPU.

17. The wearable electronic device as claimed in claim 15, wherein the device body further comprises a battery, electrically connected to the processor and configured to provide power for the processor and the FPCB.

18. An EMG strap, comprising:
two flexible silicone shells, respectively defining two accommodation cavities;
two FPCBs, respectively housed within the two accommodation cavities;
four AFE circuits, wherein two of the four AFE circuits are disposed on one of the two FPCBs, and another two of the four AFE circuits are disposed on another of the two FPCBs;
four electrode sets, wherein two of the four electrode sets are disposed on the one of the two FPCBs and respectively electrically connected to the two of the four AFE circuits, and are exposed from one of the two flexible silicone shells; and another two of the four electrode sets are disposed on the another of the two FPCBs and respectively electrically connected to the another two of the four AFE circuits, and are exposed from another of the two flexible silicone shells; and
two BTB connectors, respectively disposed on the two FPCBs, wherein the two BTB connectors are respectively exposed from two first ends of the two flexible silicone shells, one of the two BTB connectors is electrically connected to the two of the four AFE circuits disposed on the one of the two FPCBs, another of the two BTB connectors is electrically connected to the another two of the four AFE circuits disposed on another of the two FPCBs, and two second ends of the two flexible silicone shells are detachably connected to each other.

19. The EMG strap as claimed in claim 18, wherein each of the four AFE circuits comprises a preamplifier, a twin-T notch filter, a high-pass filter, a low-pass filter, and a variable gain buffer, which are sequentially electrically connected in that order; the preamplifier is electrically connected between a corresponding one of the four electrode sets and the twin-T notch filter; and the variable gain buffer is electrically connected to a corresponding one of the two BTB connectors.

20. The EMG strap as claimed in claim 19, wherein each of the two flexible silicone shells defines openings for exposing corresponding two of the four electrode sets.
